(19) United States
(12) Patent Application Publication
(10) Pub. No.: US 2019/0001161 A1
(43) Pub. Date: Jan. 3, 2019

(54) COMPOSITIONS CONTAINING POLYCARBODIIMIDE COMPOUNDS

(71) Applicant: L'OREAL, Paris (FR)

(72) Inventors: Ronak RUGHANI, Edison, NJ (US); ChoCho KHINE, Bridgewater, NJ (US)

(73) Assignee: L'OREAL, PARIS (FR)

(21) Appl. No.: 15/637,310

(22) Filed: Jun. 29, 2017

(51) Int. Cl. A61Q 5/00; A61K 8/40; A61K 8/84

(52) U.S. Cl. CPC A61Q 5/004; A61K 8/84; A61K 8/40; A61Q 5/002

(57) ABSTRACT

Disclosed are treatment compositions including a polycarbodiimide together with a carboxylic acid to enhance the quality of keratinous substrates, including keratinous fibers such as hair. In particular, compositions and methods for protecting hair from damage or adverse effects caused by extrinsic and intrinsic factors and for improving the condition of or repairing damaged hair are disclosed.

### FIELD OF THE INVENTION

The present invention relates to a composition and method for treating keratinous substrates. More particularly, the present invention relates to keratinous substrate treatment compositions having a polycarbodiimide compound, and at least one carboxylic acid, for repairing or minimizing damaging effects on keratinous substrates caused by extrinsic damage, to thereby achieve desirable or improved quality and improved mechanical properties of the keratinous substrate, in particular, keratinous fibers, and more particularly, hair.

### BACKGROUND OF THE INVENTION

The appearance and/or condition of keratinous substrates, for example, keratinous fibers such as hair, skin, nails, and lips, are often affected by both extrinsic and intrinsic factors such as aging. In particular, when keratinous substrates are exposed to environmental conditions, for example, high or low humidity or to ultraviolet radiation from the sun, these substrates can lose many of their desirable properties and even become damaged. Keratinous fibers, especially hair, are constantly exposed to harsh extrinsic conditions, such as sun, chemical damage, e.g., from detergents in shampoos, bleaching, relaxing, dyeing, and permanent waving, heat, e.g., from hair dryers or curlers, and mechanical stress or wear, e.g., from brushing or grooming activities. In addition, any type of hair can diminish in quality and/or quantity over time by age and/or due to factors such as natural greasiness, sweat, shedded skin cells from the scalp, pollution, dirt, and extreme humidity conditions.

The above-described factors can result in thinning hair and/or hair breakage and/or harm the visual appearance and the feel of the hair, and lead to lank body and decreased volume. For example, hair can dry out and lose its shine or color or become frizzy and less manageable under low and high humidity conditions. Under low humidity conditions, hair can dry out and dried-out hair tends to be less shiny and more brittle. Conversely, under high humidity conditions, hair tends to absorb water, causing hair to lose its shape and become unmanageable and unattractive. In addition, hair fibers can become less strong, thereby breaking easily under stress such as stress induced by pulling, brushing, or combing activities. Furthermore, hair can lose its desirable attributes due to physical stress on the hair such as brushing and application of heat. The magnitude of the consequences of these factors is variable, depending on, for example, the quality of the hair, length, style, and environmental factors. As such, these factors generally result in damage to the keratinous fibers, either by affecting protective materials on the surface of the hair (the cuticle), or by altering the hair fiber internally (the cortex).

More specifically, extrinsic conditions may strip protective materials from the surface of the hair, and/or they may disrupt the organized structure of the hair fibers, called the α-structure, which may be accompanied by a decrease in the tensile strength. Such damage to hair by extrinsic factors is more evident the further the hair fiber has grown from the root, because the hair has been exposed longer to such extrinsic factors. In effect, the hair has what may be called a “damage history” as it grows, i.e., the further from the root, the lower the tensile strength and the greater the breakdown in α-structure that has occurred. As a result, consumers continue to seek products such as hair care and hair cosmetic compositions which protect and enhance the appearance of hair as well as reduce the deleterious effects of adverse environmental conditions, photo-damage, and physical stress.

Morphologically, a hair fiber contains four structural units: cuticle, cortex, medulla, and intercellular cement. The cuticle layers are located on the hair surface and consist of flat overlapping cells (“scales”). These scales are attached at the root end and point toward the distal (tip) end of the fiber and form layers around the hair cortex. The cortex comprises the major part of the hair fiber. The cortex consists of spindle-shaped cells, or macrofibrils, that are aligned along the fiber axis. The macrofibrils further consist of microfibrils (highly organized protein units) that are embedded in the matrix of amorphous protein structure. The medulla is a porous region in the center of the fiber. The medulla is a common part of wool fibers but is found only in thicker human hair fibers. Finally, the intercellular cement is the material that binds the cells together, forming the major pathway for diffusion into the fibers.

The mechanical properties of hair are determined by the cortex. A two-phase model for the cortex organization has been suggested. Milczarek et al, Colloid Polym. Sci., 270, 1106-1115 (1992). In this model, water-impenetrable microfilaments (“rods”) are oriented parallel with the fiber axis. The microfilaments are embedded in a water-penetrable matrix (“cement”). Within the microfilaments, coiled protein molecules are arranged in a specific and highly organized way, representing a degree of crystallinity in the hair fiber.

Similar to other crystalline structures, hair fibers display a distinct diffraction pattern when examined by wide-angle X-ray diffraction. In normal, non-stretched hair fibers this pattern is called an “alpha-pattern”. The alpha-pattern or α-structure of hair is characterized by specific repeated spacings (9.8 Å, 5.1 Å, and 1.5 Å). All proteins that display this X-ray diffraction pattern are called α-proteins and include, among others, human hair and nails, wool, and porcupine quill. When the hair fiber is stretched in water, a new X-ray diffraction pattern emerges that is called a “β-pattern”, with new spacings (9.8 Å, 4.65 Å, and 3.3 Å).

Damage to hair may occur in the cuticle and/or the cortex. When normal hair is damaged by heat, chemical treatment, UV radiation, and/or physical/mechanical means, myriad chemical and physical changes are induced in the hair. For example, these damaging processes have been known to produce removal or damage to cuticle scales or to cleave the thioester linkage holding the hydrophobic 18-methyl eicosanoic acid (“18-MEA”) layer to hair. Thus, it is commonly observed that undamaged hair exhibits significant hydrophobic character, whereas damaged hair shows significant hydrophilic character due to the removal of surface lipids.

There is a need, therefore, for cosmetic products that are useful in protecting the chemical and physical structure of keratinous fibers from harsh extrinsic conditions and restoring the hair's physical properties to undamaged states following damage by extrinsic conditions. More particularly, there is a need to find materials or compositions or methods that can provide a protective barrier and/or treatment to hair to protect it at the cortex. Such a protective barrier or treatment should not be easily transferred from the substrate over time by normal everyday activity. As such, makers of cosmetic products such as hair and skin care products continue to seek materials and ingredients that can provide such benefits. At the same time, long lasting benefits or durability of these benefits are also desirable.

In addition, in today's market, many consumers prefer the flexibility of having products that can be used on hair or skin on different ways. Methods for caring for or non-permanent shaping of keratinous fibers include, for example, brushing, teasing, braiding, the use of hair rollers, and heat styling, optionally with a commercially available styling product. Non-limiting examples of heat styling include blow drying, crimping, curling, and straightening methods using elevated temperatures (such as, for example, setting hair in curlers and heating, and curling with a curling iron and/or hot/steam rollers and/or flat iron).

There is a need, therefore, for materials, compositions and methods that result in hair with improved quality or that is less damaged when the hair is exposed to adverse environmental and physical factors and/or when chemically treated. As such, it is also advantageous to find a means for treating damaged keratinous fibers by repairing them, that is to say by intrinsically improving the cosmetics of the keratinous fibers, reducing breakage of the keratin fibers and/or preventing breakage of the keratinous fibers. At the same time, it is desirable that said materials, compositions, treatment systems, and methods provide durable or long-lasting caring and repair benefits to hair.

To achieve at least one of these and other advantages, the present invention provides a method of protecting and/or repairing a keratinous substrate, and more particularly, a keratinous fiber chosen from hair, eyelashes and eyebrows from extrinsic damage caused by heating, UV radiation, chemical treatment or other harsh treatment by applying to said keratinous fiber a composition that includes a polycarbodiimide compound and a carboxylic acid in amounts effective to repair or reduce/prevent damage to or improve the quality of the keratinous fiber.

Another subject of the invention is the use of a polycarbodiimide compound and a carboxylic acid to improve the condition of the keratinous fibers and/or to repair damaged keratinous fibers and/or to prevent breakage of keratinous fibers.

### BRIEF SUMMARY OF THE INVENTION

In an exemplary embodiment, a keratinous fiber treatment composition including a at least one polycarbodiimide compound and at least one carboxylic acid selected from the group consisting of maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof is disclosed.

The composition includes amounts of each of the polycarbodiimide compound and the at least one carboxylic acid selected from the group consisting of maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof, sufficient to protect or repair keratinous substrates from damage or adverse effects caused by extrinsic and/or intrinsic factors, for example, before or during or after chemically treating the hair (e.g., dyeing the hair using permanent, semi-permanent or demi-permanent dyeing compositions, bleaching/lightening or lifting the color of hair by chemical oxidizing agents, perming the hair using chemical reducing/oxidizing agents, relaxing the hair using lye and no-lye compositions, straightening the hair using chemical straightening agents).

In another exemplary embodiment, the present invention is directed to a method of protecting a keratinous substrate comprising keratinous fibers chosen from hair, eyelashes and eyebrows from extrinsic damage caused by heating, UV radiation or chemical treatment, or of repairing a keratinous substrate comprising keratinous fibers chosen from hair, eyelashes and eyebrows following extrinsic damage caused by heating, UV radiation or chemical treatment. The method includes applying to the keratinous substrate a composition including the polycarbodiimide and the carboxylic acid in an amount effective to protect or repair the keratinous fibers; wherein the polycarbodiimide compound is present at a concentration of from about 0.1 to about 10% by weight, based on the total weight of the composition.

Another embodiment of the present invention is a method of protecting or repairing a keratinous substrate comprising keratinous fibers chosen from hair comprising applying to the keratinous fibers the composition of the present invention in an amount effective to protect or repair said keratinous fibers before or during or after chemically treating the hair (e.g., dyeing the hair using permanent, semi-permanent or demi-permanent dyeing compositions, bleaching/lightening or lifting the color of hair by chemical oxidizing agents, perming the hair using chemical reducing/oxidizing agents, relaxing the hair using lye and no-lye compositions, straightening the hair using chemical straightening agents).

In some embodiments, the keratinous fibers in the above described composition are heated and the composition is applied to the fibers prior to heating or during heating or after heating the fibers.

In another exemplary embodiment, a method for imparting strength to a keratinous substrate comprising at least one keratinous fiber is disclosed. The method includes applying to the at least one keratinous fiber the composition including the polycarbodiimide and the carboxylic acid.

In some embodiments, the above-described method includes a step of heating the keratinous fiber prior to or during or after the application of the above-described composition.

The present invention is also directed to a method for cosmetic treatment of keratinous substrates, such as keratinous fibers, by applying the above-disclosed composition onto a surface of the keratinous substrate, such as the cuticle of hair fibers.

The present invention is also directed to methods and kits for cosmetic treatment of keratinous substrates, such as keratinous fibers, by applying the above-disclosed composition onto a surface of the keratinous substrate, such as the cuticle of hair fibers in a step wise fashion. According to some such embodiments, the kit includes separate packaging of one or more of the actives of the inventive composition provided in one or more of thickened or un-thickened aqueous and non-aqueous phases, and packaging of any of the foregoing with one or more of processing agents selected from a coloring agent, a pigmenting agent, a permanent process agent, a relaxing process agent, a straightening process agent, and a highlighting process agent.

In particular, the keratinous fiber such as hair that is treated via the compositions, methods and systems according to the invention can result in stronger and less brittle hair and/or less hair breakage and/or in hair that remains well-behaved or is more manageable Thus, hair fibers are aligned, smooth and disentangle easily, which makes them easier to comb. The treated hair can also have more body (it is not limp) and be easier to style or shape.

Other features and advantages of the present invention will be apparent from the following more detailed description of the exemplary embodiment which illustrates, by way of example, the principles of the invention.

### BRIEF DESCRIPTION OF THE DRAWINGS

Implementation of the present technology will now be described, by way of example only, with reference to the attached figures, wherein:

FIG. 1 shows the plateau stress measurements obtained from tensile testing conducted on untreated hair fibers and hair fibers treated with test compositions, including the compositions of the present invention.

FIG. 2 shows the break stress measurements obtained from tensile testing conducted on untreated hair fibers and hair fibers treated with test compositions, including the compositions of the present invention.

FIG. 3 shows the toughness measurements obtained from tensile testing conducted on untreated hair fibers and hair fibers treated with test compositions, including the compositions of the present invention.

### DETAILED DESCRIPTION OF THE INVENTION

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“Active material” as used herein with respect to the percent amount of an ingredient or raw material, refers to 100% activity of the ingredient or raw material.

As used herein, the terms “applying a composition onto keratin fibers” and “applying a composition onto hair” and variations of these phrases are intended to mean contacting the fibers or hair, with at least one of the compositions of the invention, in any manner.

“At least one,” as used herein, means one or more and thus includes individual components as well as mixtures/combinations.

The term “comprising” (and its grammatical variations) as used herein is used in the inclusive sense of “having” or “including” and not in the exclusive sense of “consisting only of.”

“Conditioning,” as used herein, means imparting at least one of combability, manageability, moisture-retentivity, luster, shine, softness, and body to the hair.

“Durable conditioning,” as used herein, means that, following at least one shampoo/washing/rinsing after treatment of keratinous fibers such as hair with the compositions of the present disclosure, treated hair still remains in a more conditioned state as compared to untreated hair. “Heating” refers to the use of elevated temperature (i.e., above room temperature such as at or above 30° C.). In one embodiment, the heating in the inventive method may be provided by directly contacting the at least one keratinous fiber with a heat source. Non-limiting examples of heating by direct contact with the at least one keratinous fiber include flat ironing and curling methods using elevated temperatures (such as, for example, setting hair in curlers and heating, and curling with a curling iron and/or hot rollers). In another embodiment, the heating in the inventive method may be provided by heating the at least one keratinous fiber with a heat source which may not directly contact the at least one keratinous fiber. Non-limiting examples of heat sources which may not directly contact the at least one keratinous fiber include blow dryers, hair dryers, hood dryers, heating caps and steamers.

“A heat-activated” composition, as used herein, refers to a composition which, for example, imparts to at least one keratinous substrate such as a keratinous fiber, a property or a benefit or coating or film that is different or improved over the same composition or coating or film which is not heated during or before or after application of the composition. One example includes a composition which imparts better break stress and/or toughness property to at least one keratinous fiber better than the same composition which is not heated during or before or after application. Another example includes a composition that forms a coating or film on the keratinous fiber when the fiber is heated or exposed to elevated temperatures.

“High humidity,” as defined herein, refers to atmospheric humidity above 40%.

“Homogeneous” means having the visual appearance of being substantially uniform throughout, i.e., visually appears as a single-phase emulsion and/or dispersion.

“Keratinous substrate,” as used herein, includes, but is not limited to, skin, hair, and nails. “Keratinous substrate” as used herein also includes “keratinous tissue” or “keratinous fibers,” which as defined herein, may be human keratinous fibers, and may be chosen from, for example, hair, such as hair on the human head, or hair comprising of eyelashes or hair on the body.

The term “style” or styling” as used herein includes shaping, straightening, curling, or placing a keratin fiber such as hair, in a particular arrangement, form or configuration; or altering the curvature of a keratinous fiber or other substrate; or re-positioning a keratin fiber or other substrate to a different arrangement, form or configuration; or providing/maintaining a hold to the shape or configuration of the keratin fiber. In some embodiments, the hold to the shape of configuration of the fiber may be expressed as an improved bending force property.

The term “treat” (and its grammatical variations) as used herein refers to the application of the compositions of the present invention onto keratinous substrates such as keratinous fibers or hair or skin or to contacting said keratinous substrates with the compositions of the present invention.

The term “repair” (and its grammatical variations) as used herein means that the damaged keratinous fibers such as hair fibers following treatment with the compositions of the present disclosure showed an improvement in tensile properties or strength which are similar to that of natural undamaged hair. The improvement in tensile properties can be determined or assessed by any available means such as by mechanical tests of the fibers and/or by consumer and sensory evaluations of perceivable fiber strengthening and/or physical attributes which have a positive impact on fiber mechanical properties.

The term “wash cycle” as used herein, refers to a step or process of washing a keratinous substrate and may include treating the substrate with a surfactant-based product (e.g., shampoo or conditioner or body wash) then washing or rinsing the substrate with water. The term “wash cycle” may also include washing or rinsing the substrate with water.

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

It is an object of the present invention to provide materials and compositions and methods which provide both a protective barrier or repairing treatment onto keratinous substrates such as hair and which impart native/undamaged physical properties—such as strength, repair, less breakage, ease of combing, conditioning, etc.—to hair, in particular, damaged hair, as well as impart physical properties mimicking natural/undamaged hair to damaged hair.

It is also an object of the present invention to provide materials and compositions and methods which protect or repair a keratinous fiber chosen from hair comprising applying to the keratinous fiber the composition of the present invention in an amount effective to protect or repair said keratinous fiber before or during or after chemically treating the hair (e.g., dyeing the hair using permanent, semi-permanent or demi-permanent dyeing compositions, bleaching/lightening or lifting the color of hair by chemical oxidizing agents, perming the hair using chemical reducing/oxidizing agents, relaxing the hair using lye and no-lye compositions, straightening the hair using chemical straightening agents).

As such, it is desirable to formulate hair repair strategies to address the need for both surface and cortex repair. The present invention directed to compositions, methods and agents employing a carbodiimide polymer and a carboxylic acid was found to provide mechanical strength to the fiber that yields a repaired/reinforced fiber that is able to withstand day-to-day grooming.

In an embodiment, the present disclosure is directed to a composition for the treatment of keratinous fibers such as hair, comprising:

at least one polycarbodiimide compound, present in a concentration, by weight, of from about 0.1% to about 10%, or from about 0.2% to about 5%, or from about 0.3% to about 3%, based on the total weight of the composition; and at least one carboxylic acid selected from the group consisting of maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof, and present in a concentration, by weight, of from about 0.25% to about 20%, or from about 0.4% to about 10%, or from about 0.5% to about 5%, based on the total weight of the composition.

In an embodiment, the at least one polycarbodiimide compound in the above-described composition has a formula:

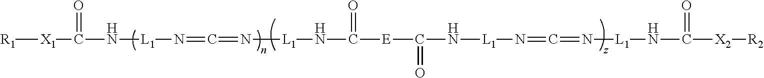

wherein X₁ and X₂, each independently, represents O, S or NH; R₁ and R₂, each independently, a hydrocarbon group containing one or more catenary or non-catenary hetero-atoms and containing linear or branched and cyclic or acyclic groups which are ionic or non-ionic segments or a partially or fully fluorinated hydrocarbon group containing one or more catenary or non-catenary hetero-atoms; n and z are, each independently, an integer of 0 to 20; L₁ represents a C₁ to C₁₈ divalent aliphatic hydrocarbon group, a C₃ to C₁₃ divalent alicyclic hydrocarbon group, a C₆ to C₁₄ divalent aromatic hydrocarbon group, a C₃ to C₁₂ divalent heterocyclic group, or a C₆ to C₁₄ divalent aromatic hydrocarbon group, wherein a plurality of L₁ groups may be identical to or different from one another; E is a radical selected from:

O−R₃—O; S—R₄—S; and

R₅—N—R₄—N—R₅;

wherein R₃ and R₄ are, each independently, hydrocarbon radicals that may contain halogen atoms or one or more catenary or non-catenary hetero atoms, including an aromatic, cycloaliphatic, aryl and linear or branched alkyl radical and R₅ is hydrogen or a hydrocarbon radical, the hydrocarbon radical, when present, includes halogen atoms or one or more catenary or non-catenary hetero atoms.

In an embodiment, the at least one carboxylic acid in any one of the above-described composition is chosen from maleic acid, oxalic acid, malonic acid, malic acid, citric acid, glycolic acid, and mixtures thereof, and is preferably chosen from maleic acid, oxalic acid, malonic acid, citric acid, and mixtures thereof, and is more preferably chosen from maleic acid.

In an embodiment, at least one carboxylic acid is present in an equal proportion to or greater proportion than the polycarbodiimide compound.

In an embodiment, the present disclosure is directed to a method of treating hair, the method comprising applying any one of the above-described compositions onto the hair.

In an embodiment, the present disclosure is directed to a hair treatment agent comprising a first component containing the at least one polycarbodiimide compound and a solvent; and a second component containing the at least one carboxylic acid and a solvent; wherein the first and the second components are each contained in separate containers and wherein the first and the second components are applied on hair in a step-wise manner. The solvent is chosen from water, organic solvents, and mixtures thereof.

It has been surprisingly and unexpectedly discovered by the inventors that a composition containing the combination of carboxylic acids selected from the group consisting of maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof, and polycarbodiimide compounds, when applied to keratinous substrates such as hair, enhance the properties of hair wherein the combination increases the conditioning effect (e.g., smoothness, less damaged feel), strengthens or improves the mechanical properties of the hair (such as less or no breakage, greater tensile strength), increases the stiffness and humidity resistance of hair and ameliorates the condition of damaged hair by improving the appearance and quality of hair (for example, smoother feel, softer feel, less damaged feel, more discipline).

Without being bound to any one theory, the inventors of the present disclosure believe that the polycarbodiimide compounds and the carboxylic acids selected from the group consisting of maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof comprising the keratinous substrate treatment compositions of the invention react with each other and to the keratinous substrate via crosslinking reactions when such compositions are applied onto keratinous substrates such as hair or skin. It is also believed that exposing a keratinous substrate such as hair that has been contacted with the compositions of the invention to heat or temperatures above room temperature enhance the kinetics of the crosslinking reactions resulting into the cosmetic and fiber repair/strengthening attributes imparted to the hair.

Thus, the compositions of the present disclosure can provide a cosmetic treatment such as hair care, hair treatment, and hair styling/shaping products such that the quality of the hair is improved or restored resulting in significantly better cosmeticities, feel and appearance, and less damaged condition of the hair.

The compositions according to the invention, are compositions including polycarbodiimide compounds and carboxylic acids selected from the group consisting of maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof. The composition may include other suitable ingredients for hair treatment or hair repair. For example, known solvents and/or additives may be utilized in addition to the polycarbodiimide compounds and carboxylic acids to provide additional benefits to the composition. The range of concentrations by weight of the composition over which the association provides caring and/or repairing benefits to keratinous substrates is about 0.35% to about 30% total actives.

In various embodiments, the weight ratio of the at least one carboxylic acid to the polycarbodiimide compound over which the association provides caring and/or repairing benefits to keratinous fibers can range from about 10:1 to 1:2 or about 9:1 to about 1:1 or about 8:1 to about 1:1 or about 7:1 to about 1:1 or about 6.5:1 to about 1:1 or about 6.25 to about 1;1 or about 6:1 to about 1:1 or about 5:1 to about 1:1 or about 4:1 to about 1:1 or about 3:1 to about 1:1 or about 2:1 to about 1:1, including ranges and sub-ranges there between.

In certain embodiments, the weight ratio of the at least one carboxylic acid to the polycarbodiimide compound is at about 10, 9, 8, 7, 6, 6:25, 6, 5.5, 6, 5, 4, 3, 2, 1, 0.9, 0.8, 0.7, 0.6, or 0.5.

A carbodiimide group is a linear triatomic moiety generally depicted by Formula (I):

*—(N═C═N)—* (I)

At least one of the nitrogens is linked to or incorporated into a backbone or other bridging group to result in a molecule having at least two carbodiimide groups.

Polycarbodiimides

In one embodiment, the polycarbodiimides comprising of at least two carbodiimide units, as described above, can be represented by Formula (II):

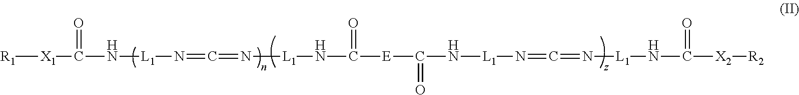

wherein X1 and X2 each independently represent O, S or NH. R1 and R2 are selected from a hydrocarbon group containing one or more catenary or non-catenary hetero-atoms, such as nitrogen, sulfur and oxygen, and linear or branched and cyclic or acyclic groups which can be ionic or non-ionic segments, or a partially or fully fluorinated hydrocarbon group that may contain one or more catenary or non-catenary hetero-atoms; n and z are, each independently, an integer of 0 to 20; L1 (Linker of carbodiimide groups) is selected from a C1 to C18 divalent aliphatic hydrocarbon group, a C3 to C13 divalent alicyclic hydrocarbon group, a C6 to C14 divalent aromatic hydrocarbon group, and a C3 to C12 divalent heterocyclic group; wherein a plurality of L1s may be identical to or different from one another, and wherein in another embodiment, L1 of formula (II) is selected from a C1 to C18 divalent aliphatic hydrocarbon group, a C3 to C13 divalent alicyclic hydrocarbon group, a C6 to C14 divalent aromatic hydrocarbon group, and a C3 to C12 divalent heterocyclic group; wherein a plurality of L1s may be identical to or different from one another;

wherein E is a radical selected from the following formulas:

O—R3-O; S—R4-S; and

R5-N—R4-N—R5;

wherein R3 and R4 are each independently hydrocarbon radicals that may contain halogen atoms or one or more catenary (i.e.; in chain, bonded only to carbon) or non-catenary hetero atoms, including an aromatic, cycloaliphatic, aryl and alkyl radical (linear or branched) and R5 is hydrogen, or a hydrocarbon radical which can contain halogen atoms or one or more catenary (i.e.; in chain, bonded only to carbon) or non-catenary hetero atoms.

Examples of R1 and R2 can be methyl glycolate, methyl lactate, polypropylene glycol, polyethylene glycol monomethyl ether, dialkylamino alcohol.

Examples of L1 can be the diradical of tolylene, hexamethylene, hydrogenated xylylene, xylylene, 2,2,4-trimethylhexamethylene, 1,12-dodecane, norbornane, 2,4-bis-(8-octyl)-1,3-dioctylcyclobutane, 4,4′-dicyclohexylmethane, tetramethylxylylene, isophorone, 1,5-naphthylene, 4,4′ diphenylmethane, 4,4′ diphenyldimethylmethane, phenylene.

Polycarbodiimides may include polymers with a plurality of carbodiimide groups appended to the polymer backbone. For example, U.S. Pat. No. 5,352,400 (the disclosure of which is incorporated by reference herein for all purposes as if fully set forth) discloses polymers and co-polymers derived from alpha-methylstyryl-isocyanates. Such a polymer is illustrated in Formula (III).

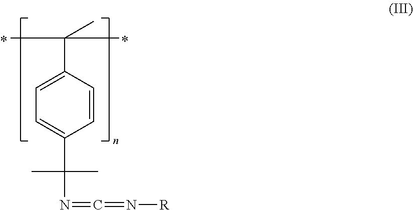

wherein R is an alkyl, cycloalkyl or aryl group (in some particular embodiments having from 1 to 24 carbon atoms).

In another embodiment, polycarbodiimides, according to the present disclosure, include polycarbodiimides having branched structures, like that shown in Formula (IV), and as described in Chapter 8 of Technology for Waterborne Coatings, E. J. Glass Ed., ACS Symposium 663, 1997; The Application of Carbodiimide Chemistry to Coating, by J. W. Taylor and D. R. Bassett (the disclosure of which is incorporated by reference herein for all purposes as if fully set forth).

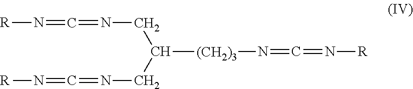

wherein R is an alkyl, cycloalkyl or aryl group (in some particular embodiments having from 1 to 24 carbon atoms).

An example of a polycarbodiimide compound that has a linker L1 chosen from m-tetramethylxylylene is an aromatic functionalized polycarbodiimide according to the following formula (V):

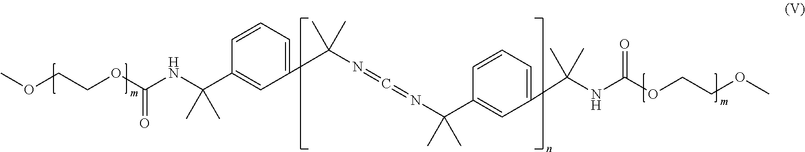

wherein m and n, each independently, are selected from integers from 1 to 100.

In one embodiment, the compositions of the present disclosure do not employ a polycarbodiimide compound of formula (V) above.

In one embodiment, the polycarbodiimide compound of the present disclosure is not chosen from the compound of formula (V) above.

Suitable polycarbodiimide compounds include, but are not limited to, those commercially sold by the suppliers Nisshinbo, Picassian, and 3M. Particularly suitable polycarbodiimide compounds include, but are not limited to, those known by the name under the CARBODILITE series, V-02, V02-L2, SV-02, E-02, V-10, SW-12G, E-03A, commercially sold by Nisshinbo.

In some embodiments, the polycarbodiimide of the present disclosure is selected from compounds of formula (II) wherein L1 (Linker of carbodiimide groups) represents a C1 to C18 divalent aliphatic hydrocarbon group, a C3 to C13 divalent alicyclic hydrocarbon group, a C3 to C12 divalent heterocyclic group, or a C6 to C14 divalent aromatic hydrocarbon group;

wherein a plurality of L1s may be identical to or different from one another.

The polycarbodiimide compound is typically present in the composition of the present disclosure in an amount of from about 0.1% to about 10%, by weight, in some particular embodiments from about 0.2% to about 5%, by weight, or from about 0.3% to about 3%, by weight, including all ranges and subranges therebetween, based on the total weight of the composition.

In various embodiments, the amount of the polycarbodiimide compound in the composition of the present disclosure is about 0.1%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 0.975%, 1%, 1.1%, 1.2%, 1.4%, 1.5%, 2%, 2.5%, 3%, 3.5%, 4%, 4.5%, 5%, 5.5%, 6%, 6.5%, 7%, 7.5%, 8%, 8.5%, 9%, 9.5%, and 10%, by weight, by weight, based on the total weight of the composition.

Carboxylic Acids

The at least one carboxylic acid of the present invention is selected from the group consisting of maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof.

The at least one carboxylic acid of the present invention will typically be present in the composition of the present disclosure in an amount of from about 0.25% to about 20%, by weight, in some particular embodiments from about 0.4% to about 10%, by weight, and in some particular embodiments from about 0.5% to about 5.0%, or from about 0.02% to about 5.0%, or from about 0.05% to about 4%, or from about 0.1% to about 3%, by weight, including all ranges and subranges therebetween, based on the total weight of the composition.

In various embodiments, the amount of the carboxylic acid in the composition of the present disclosure is about 0.02%, 0.03%, 0.04%, 0.05%, 0.08%, 0.1%, 0.2%, 0.25%, 0.5%, 0.55%, 0.9%, 1%, 1 .5%, 2%, 2.5%, 3%, 3.5%, 4%, 4 .5%, 5%, 5.5%, 6%, 6.5%, 7%, 7.5%, 8%, 8.5%, 9%, 9.5%, 10%, 12%, 14%, 15%, 16%, 18%, and 20% by weight, based on the total weight of the composition.

Solvent

The composition, according to the present disclosure, further includes suitable solvents for treatment of keratinous fibers. Examples of suitable solvents include water, in some particular embodiments distilled or de-ionised, or organic solvents or mixtures thereof.

Suitable organic solvents may be chosen from volatile and nonvolatile organic solvents.

Suitable organic solvents are typically C1-C4 lower alcohols, glycols, polyols, polyol ethers, hydrocarbons, and oils. Examples of organic solvents include, but are not limited to, ethanol, isopropyl alcohol, benzyl alcohol, phenyl ethyl alcohol, propylene glycol, pentylene glycol, hexylene glycol, glycerol, and mixtures thereof.

Other suitable organic solvents include glycol ethers, for example, ethylene glycol and its ethers such as ethylene glycol monomethyl ether, ethylene glycol monopropyl ether, ethylene glycol monobutyl ether, propylene glycol and its ethers, such as propylene glycol monomethyl ether, propylene glycol monopropyl ether, propylene glycol monobutyl ether, dipropylene glycol and diethylene glycol alkyl ethers, such as diethylene glycol monoethyl ether, diethylene glycolmonobutyl ether, and dipropylene glycol n-butyl ether. Glycol ethers are commercially available from The Dow Chemical Company under the DOW E-series and DOW P-series. In an exemplary embodiment, glycol ether for use in the present invention is dipropylene glycol n-butyl ether, known under the tradename of DOWANOL DPnB.

Suitable organic solvents also include synthetic oils and hydrocarbon oils include mineral oil, petrolatum, and C10-C40 hydrocarbons which may be aliphatic (with a straight, branched or cyclic chain), aromatic, arylaliphatic such as paraffins, iso-paraffins, isododecanes, aromatic hydrocarbons, polybutene, hydrogenated polyisobutene, hydrogenated polydecene, polydecene, squalene, petrolatum and isoparaffins, silicone oils, fluoro oils and mixtures, thereof.

The term “hydrocarbon based oil” or “hydrocarbon oil” refers to oil mainly containing hydrogen and carbon atoms and possibly oxygen, nitrogen, sulfur and/or phosphorus atoms. Representative examples of hydrocarbon based oils include oils containing from 8 to 16 carbon atoms, and especially branched C8 C16 alkanes (also known as isoparaffins), for instance isododecane (also known as 2,2,4,4,6 pentamethylheptane), isodecane and isohexadecane.

Examples of silicone oils that may be useful in the present invention include nonvolatile silicone oils such as polydimethylsiloxanes (PDMS), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2 phenylethyl trimethylsiloxysilicates, and dimethicones or phenyltrimethicones with a viscosity of less than or equal to 100 cSt.

Other representative examples of silicone oils that may be useful in the present invention include volatile silicone oils such as linear or cyclic silicone oils, and especially containing from 2 to 10 silicon atoms and in particular from 2 to 7 silicon atoms, these silicones optionally comprising alkyl or alkoxy groups containing from 1 to 10 carbon atoms. Specific examples include dimethicones with a viscosity of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Representative examples of fluoro oils that may be suitable for use in the present invention include volatile fluoro oils such as nonafluoromethoxybutane and perfluoro methylcyclopentane.

Particularly suitable solvents in the composition of the present disclosure include water, isododecane, ethanol, and combinations thereof. The solvent will typically be present in total amounts ranging from about 60% to 98%, in some particular embodiments from 80% to 96%, by weight, including all ranges and subranges therebetween, based on the total weight of the composition.

In yet some other embodiments, the solvent of the present disclosure does not comprise water and/or organic solvent that is added as a separate ingredient, by itself, into the compositions of the present invention, such that water and/or organic solvent is present in the compositions of the present invention when it accompanies one or more ingredients of a raw material that is added into the compositions of the invention.

When the compositions of the disclosure contain water, according to various embodiments, water can be present in amounts of about 98% or less, such as about 96%, 95%, 90%, 85%, 80%, 75%, 70%, 65%, 60%, 55%, 50%, 48%, 46%, 45%, 44%, 42%,40%, 35%,30%, 20%, 10%, or 5% or less, by weight, based on the total weight of the composition.

When the compositions of the disclosure contain an organic solvent(s), according to various embodiments, the organic solvent(s) can be present in a total amount of about 98% or less, such as about 96%, 95%, 90%, 85%, 80%, 75%, 70%, 65%, 60%, 55%, 50%, 48%, 46%, 45%, 44%, 42%, 40%, 35%, 30%, 20%, 10%, or 5% or less, by weight, based on the total weight of the composition.

Additives

The composition, according to the present disclosure, further includes suitable additives for treatment of keratinous fibers.

The composition according to the disclosure may also comprise additives chosen from surfactants (anionic, nonionic, cationic and amphoteric/zwtterionic), rheology modifiers, thickening and/or viscosity modifying agents, nacreous agents, opacifiers, fragrances, vitamins, preserving agents, pH stabilizers, and mixtures thereof.

The compositions of certain embodiments may comprise stabilizers, for example sodium chloride, magnesium dichloride or magnesium sulfate.

If present in the composition, the above-described additives are generally present in an amount ranging up to about 95% by weight including all ranges and subranges therebetween, based on the total weight of the composition, such as up to about 50%, up to about 40%, up to about 30%, up to about 20%, up to about 15%, up to about 10%, up to about 5%, such as from about 0.001% to about 50%, or from about 0.001% to about 40%, or from about 0.001% to about 30%, or from about 0.001% to about 20%, or from about 0.001% to about 10%, by weight, based on the total weight of the composition.

Needless to say, a person skilled in the art will take care to select this or these optional additional compound(s), and/or the amount thereof, such that the advantageous properties of the composition, according to the invention, are not, or are not substantially, adversely affected by the envisaged addition.

Methods of Preparation and Methods of Use

In some embodiments, the compositions of the present disclosure are prepared by combining the polycarbodiimide compound, the carboxylic acid, and a solvent.

In other embodiments, the compositions of the present disclosure are prepared by combining the polycarbodiimide compound, the carboxylic acid, and a solvent chosen from water, organic solvents, and mixtures thereof.

In yet other embodiments, the compositions of the present disclosure are prepared by combining the polycarbodiimide compound, the carboxylic acid, and a solvent comprising water and organic solvents.

One-Step Application Process

In one embodiment, the composition of the present disclosure is applied onto keratinous fibers such as hair in a one-step application process. The composition for use in the one-step application is prepared by combining the ingredients, including the polycarbodiimide compound, the carboxylic acid, and solvent, resulting in a composition that is then applied onto the keratinous fibers.

Two-Step Application Process

In other embodiments, the composition of the present disclosure is applied onto keratinous fibers such as hair in a two-step application process. In a two-step application process, the individual components (polycarbodiimide compound combined with a solvent and carboxylic acid combined with a solvent) are applied to the keratinous fibers in a step-wise fashion in any order to treat the fibers.

The application of the composition onto a keratinous fibers such as hair, according to the present disclosure, may occur at room temperature.

In other embodiments, the application of the composition according to the present disclosure, may occur at an elevated temperature (or temperatures greater than room temperature) by applying heat to the hair or exposing hair to elevated temperatures. While not so limited, heating may be provided, for example, by commonly used heating tools for example a helmet dryer or blow dryer (about 30° C. and above, such as from about 30° C. to about 100° C. or from about 40° C. to about 80° C. or from about 40° C. to about 70° C. or from about 50° C. to about 70° C.) or hot iron or flat iron (about 130° C. about 250° C.) or steam/hot rollers.

In an embodiment, the method of treating hair involves the general protocol (I) of:
(a) treating the hair with the compositions of the present invention according to a one-step or a two-step application process (hair can be pre-washed or pre-shampooed before treatment); and
(b) optionally, allowing the composition to remain on the hair for at least 30 minutes (up to about 60 minutes) at an elevated temperature or at any one of the above-described temperature ranges before shampooing or washing the hair.

In another embodiment, the method of treating hair involves the general protocol (II) of:
(a) treating the hair with the compositions of the present invention according to a one-step or a two-step application process (hair can be pre-washed or pre-shampooed before treatment);
(b) combing the hair and heating the hair such as by flat-ironing the hair using a flat-iron device or by blow drying the hair using a blow dryer device, wherein the flat-iron can be set at a temperature ranging from about 135° C. about 250° C. or from about 150° C. about 200° C. or from about 160° C. about 190° C., or from about 170° C. about 180° C., and preferably at about 177° C. (350° F.) and the blow dryer can be set at a temperature ranging from about 30° C. about 100° C. or from about 35° C. about 80° C. or from about 40° C. about 70° C., or from about 45° C. about 60° C.; and
(c) optionally, allowing the composition to remain on the hair for at least 30 minutes (up to about 60 minutes) at an elevated temperature or at any one of the above-described temperature ranges before shampooing or washing the hair.

According to at least one embodiment, the above-described methods comprise applying to the hair, an effective amount of any one of the compositions of the present disclosure.

The compositions of the present disclosure may be employed in an effective amount to adequately cover the surface of the fibers of the hair and to achieve the desired effects of hair repair and conditioning.

An effective amount of the composition is typically from about 0.1 gram to about 50 grams per head of hair, and in some applications for treatment of hair, in amounts from about 20 to 60 grams, and in yet further embodiments for an abundance of hair in amounts from about 20 to about 80 grams or more. It will thus be appreciated that the amounts applied depend on the amount or volume of keratinous substrates, such as hair, to be treated and may thus fall within lower ranges for small amounts or patches of hair to the higher ranges and beyond for large amounts or patches of hair. Typical applications are to the whole head in the case of treatment of hair. It will be understood that application to the hair typically includes working the composition through the hair.

Further disclosed herein is the use of the compositions of the present disclosure for caring for the hair such as for hair repair treatments, or for reducing damage to the hair or for improving the manageability of the hair.

The compositions may be applied to wet or dry hair, before or after shaping. They may be used in a non-rinse fashion. In some other embodiments, the composition may be rinsed from the hair.

In an embodiment, the hair that has been contacted with the compositions of the present invention is further heated or exposed to elevated temperatures (above room temperature). The heat source can be chosen from a blow dryer, a flat iron, a hair dryer, a heat lamp, a heat wand, or other similar devices.

A smoothing or crimping or other mechanical action may be applied on the hair while heating the hair by use of suitable devices, including a hair brush, comb, or flat iron. The smoothing action on the hair may also include running the fingers through the hair.

A suitable applicator device for contacting the hair with compositions of the present invention is an applicator brush. It will be appreciated that while a brush is an example of a suitable applicator, particularly for hair, other applicators may be used, including but not limited to spray bottles, squeeze bottles, one and two chamber pumps, tubes, combs, and other applicators known in the art.

In addition, independently of the embodiment use, the composition present on the fibers or hair is left in place for a time, generally, from about 1 to about 60 minutes, such as from about 5 to about 45 minutes, or such as from about 5 to about 20 minutes, or such as from about 10 to about 20 minutes, or such as of about 20 minutes or such as of about 10 minutes. In alternate embodiments, the treatment times may be longer, and in some embodiments, appreciably longer, such that the application may be left on for up to 24 hours to about 48 hours.

The compositions of the present invention are easy to spread on hair.

It has surprisingly and unexpectedly discovered that the application of the composition onto hair results in improved mechanical strength or repaired split ends, or more conditioned hair. It was also surprisingly and unexpectedly discovered that the application of the composition of the present invention onto the hair resulted in improved hair quality, for example, one or more of better hair feel, less damaged feel, smoother hair, better hair manageability, or improved appearance of the hair (e.g., healthy look).

The composition, according to the present disclosure, when applied to hair, provides a strengthening and repair benefit to the hair which can be determined from tensile testing of the hair fibers based on the parameters of plateau stress, break stress and toughness values. Increases in these values indicate that the hair fiber is stronger or became stronger/reinforced (requires more force to break) when treated according to the compositions and the methods of the present invention.

In one embodiment, the plateau stress on hair fibers treated according to the compositions and the methods of the present invention is equal to or greater than 100 MPa or equal to or greater than 110 MPa and can range from about 100 to about 160 MPa, such as from about 110 to about 158 MPa or from about 115 to about 155 MPa or from about 117 to about 154.3 MPa, including all ranges and sub-ranges therebetween. With heat-activation or application of heat on the treated fibers, the plateau stress is equal to or greater than 125 MPa or equal to or greater than 130 MPa and can range from about 130 to about 220 MPa, such as from about 135 to about 210 MPa or from about 140 to about 200 MPa or from about 148.6 to about 198.4 MPa.

In one embodiment, the break stress on hair fibers treated according to the compositions and the methods of the present invention is equal to or greater than 120 MPa or equal to or greater than 140 MPa and can range from about 120 to about 250 MPa, such as from about 130 to about 245 MPa or from about 140 to about 240 MPa or from about 159.07 to about 236.8 MPa, including all ranges and sub-ranges therebetween. With heat-activation or application of heat on the treated fibers, the plateau stress is equal to or greater than 160 MPa or equal to or greater than 180 MPa and can range from about 160 to about 340 MPa, such as from about 180 to about 330 MPa or from about 190 to about 320 MPa or from about 201.6 to about 303.9 MPa.

In one embodiment, the toughness property of hair fibers treated according to the compositions and the methods of the present invention is equal to or greater than 3 10⁷ J/m³ or equal to or greater than 5 10⁷ J/m³ and can range from about 3 to about 10 10⁷ J/m³, such as from about 3.5 to about 9.5 10⁷ J/m³ or from about 4 to about 9 10⁷ J/m³ or from about 4.46 to about 8.06 10⁷ J/m³, including all ranges and sub-ranges therebetween With heat-activation or application of heat on the treated fibers, the plateau stress is equal to or greater than 4 10⁷ J/m³ or equal to or greater than 4.5 10⁷ J/m³ and can range from about 4 to about 12 10⁷ J/m³, such as from about 4.5 to about 11 10⁷ J/m³ or from about 4.8 to about 11.5 10⁷ J/m³ or from about 5.0 to about 10.6 10⁷ J/m³.

The hair care and hair repair effects obtained using the compositions and methods of the present invention may also be durable or long-lasting, i.e., wash or shampoo resistant.

Instructions for applying the composition of the present invention onto keratinous substrates such as hair on the head or eyelashes or skin may comprise directions of use of the composition for the end-user to follow. The end-user may be a consumer or cosmetologist or salon hair dresser. Directions may comprise instructing the end-user to take an amount of the composition in sufficient quantity such that the composition adequately covers the substrates and imparts the desired effects. Directions may additionally instruct the end-user to use a device (e.g., heating device) to achieve the desired effects. Instructions for using the compositions of the present invention may appear on the container (such as can, bottle or jar) holding the composition of the present invention or on the box or carton or other packaging comprising the container holding the composition.

The compositions described above are useful for application onto keratinous fibers such as hair on the head of human individuals.

Thus, the compositions of the present invention can be made into various cosmetic products such hair care hair treatment, and hair styling products.

Representative types of hair care compositions, including hair care and styling compositions, of the present invention include compositions for conditioning or protecting hair from heat damage, leave-in hair treatments, rinse-off hair treatments, combination shampoo/styling compositions, hair volumizing compositions, compositions for shaping the hair or maintaining the shape of the hair or styling products (e.g., gels, creams, milks, pastes, waxes, ointments, serums, foams, hair lotions, mousses, pump-sprays, non-aerosol sprays and aerosol sprays).

The compositions of the present invention can be in the form of an aqueous composition or an emulsion, such as a lotion or cream, and in some embodiments may be applied in another form, such as in a serum such as an anhydrous serum (substantially free of water, for example containing less than 0.1% water or not containing added water to the composition).

In one embodiment, the composition of the present invention is in the form of a non-aerosol spray, and in some particular embodiments containing a volatile organic solvent/compound.

In one embodiment, the composition of the present invention is in the form of a cream.

The compositions may be packaged in various forms, especially in a tube, a jar or bottles, in pump bottles, in squeeze bottles, or in aerosol containers so as to apply the composition in vaporized form or in the form of a mousse. The compositions may also impregnate applicators, especially gloves or wipes.

The composition may be applied by hand, with an applicator nozzle or actuator pump, with a container equipped with a pump, an applicator and a dispensing comb, or with an insoluble substrate impregnated with the composition.

As used herein, the process and composition disclosed herein may be used on the hair that has not been artificially dyed, pigmented or permed.

As used herein, the process and composition disclosed herein may be also used on the hair that has been artificially dyed, pigmented or permed, relaxed, straightened or other chemical process.

The compositions according to the disclosure may be prepared according to techniques that are well known to those skilled in the art.

Although the foregoing refers to various exemplary embodiments, it will be understood that the disclosure is not so limited. It will occur to those of ordinary skill in the art that various modifications may be made to the disclosed embodiments and that such modifications are intended to be within the scope of the disclosure. Where an embodiment employing a particular structure and/or configuration is illustrated in the present disclosure, it is understood that the present disclosure may be practiced with any other compatible structures and/or configurations that are functionally equivalent provided that such substitutions are not explicitly forbidden or otherwise known to be impossible to one of ordinary skill in the art.

The following examples are intended to further illustrate the present invention. They are not intended to limit the invention in any way. Unless otherwise indicated, all parts are by weight.

### EXAMPLES

The following examples are to illustrate the invention and are non-limiting. In accordance with the various examples, the evaluations and results demonstrate generally that the inventive compositions and methods confer beneficial results, including improved mechanical strength and quality of hair.

Procedure for preparation of compositions for application onto hair

The compositions were generated by weighing the active RM(s) (raw material) into vials and stirring the RM(s) in the respective solvent (for example, water or organic solvent or mixtures thereof). The vials were shaken vigorously by hand for 10 seconds/g of solution to create an aqueous solution. 0.4 g of product was applied to 1 g of hair. In the case of 2-step treatments, designated or desired amounts of the stock solutions were applied directly to hair without pre mixing.

TABLE 1
Representative Actives

| Generic name | Ingredient Name or Trade Name |
|---|---|
| Carboxylic acid 1 | Maleic acid or Cis-butenedioic acid, available |
| | from the supplier, Sigma-Aldrich |
| Polycarbodiimide 1 | V-02-L2 (“CDI”), Nisshinbo |

TABLE 2
Compositions

| | Composition A | Composition B | Composition C |
|---|---|---|---|
| Composition | (% by weight)* | (% by weight)* | (% by weight)* |
| Carboxylic acid 1 | 2.5 | — | 2.5 |
| (MA) | | | |
| Polycarbodiimide 1 | — | 0.4 | 0.4 |
| (CDI) | | | |
| Water | q.s to 100 | q.s to 100 | q.s to 100 |

*% by weight of active material

Protocols for Treating Hair
Protocol A. Treatment with the test compositions (30 min at elevated temperature+48 hours at ambient temperature and elevated humidity conditions). The following steps were followed:
1) One gram bleached hair swatches (highly bleached hair) were washed once with a conventional shampoo in a 1:0.4 hair to shampoo weight ratio by wetting the hair with water for 5 seconds, then treating the hair with shampoo for 30 seconds, and then rinsing the hair with water for 30 seconds After the hair was washed, the hair was wiped with a paper towel to remove excess water from the hair. The hair was damp but no water dripped. 0.4 gm of the inventive or comparative composition was applied to the hair using a plastic pipette, and the hair was combed well so that the composition was evenly distributed
2) The treated hair swatches were placed in a 50° C. oven for 30 min
3) The hair swatches were removed from the oven and placed in a 23±1° C. and 45±1% relative humidity (RH) environmental chamber for 48 hours.
4) Then the hair swatches were rinsed with warm water to remove excess solution and washed with the conventional shampoo once, then rinsed with water.
5) The hair swatches were blot dried with paper towel and dried with a blow drier set at about 70° C. for a minute.
6) After drying the hair swatches, they were placed in 23±1° C. and 45±1% RH environmental chamber for at least 8 hours prior to assessing the hair quality (by mechanical and/or cyclic fatigue testing).
Protocol B. Treatment with inventive and comparative compositions (30 min at elevated temperature+flat iron treatment+48 hours at ambient temperature and elevated humidity conditions). The following steps were followed:
1) Steps 1-3 from Protocol A were followed.
2) The hair swatches were gently combed and then flat-ironed using a flat-iron device (350° F.), with 4 passes of the flat-iron over each swatch.
3) Then, the hair swatches were placed in a 23±1° C. and 45±1% RH environmental chamber for 48 hours.
4) The hair swatches were rinsed with warm water to remove excess solution and washed with a conventional shampoo once, then rinsed with water.
5) The hair swatches were blot dried with paper towel and dried with a blow drier set at about 70° C. for a minute.
6) After drying the hair swatches, they were placed in 23±1° C. and 45±1% RH environmental chamber for at least 8 hours prior to assessing the hair quality (by mechanical and/or cyclic fatigue testing).

Performance Assessments by Mechanical Property Tests Before and After Treatment of Hair with the Test Compositions in Reference to Untreated Hair.

General Protocol

The cross-sectional area of 50 fibers from each swatch was evaluated using a Laser Scanning Micrometer Fdas770 from Dia-Stron Limited. The values were determined by taking the average of 5 cross-sectional areas along 3 cm length of the fiber. Tensile properties were measured using a Dia-Stron Miniature Tensile Tester, MTT 686, extending the fibers at a rate of 15mm/min. All tests were performed after the fibers equilibrated in the environmental chamber at 23±1° C. and 44±2% RH. Data was analyzed using the UV Win software.

Statistical Analysis: T

A one-way ANOVA analysis was performed using Tukey-Kramer statistical analysis to determine differences between controls and treated samples. Statistical significance is recognized at p<0.05.

Parameters Measured:

I. Plateau Stress Measurements—Plateau stress is a measure of the α-structure deformation of the fiber. Yield and plateau deformation are associated with molecular transition and measured in MPa. A significant increase of plateau stress values indicates that the fiber has a greater energy absorption capability before failure (breakage).

II. Break Stress Measurements—Break stress is a measure of the force required to break the fiber as measured in MPa. Significantly increased break stress values indicate reinforce hair cortex of bleach damaged hair.

III. Toughness Measurements—The toughness parameter is a measure of how strong and resistant the fiber is to breakage and is measured in J/m³.

An increase in toughness of the fiber indicates a reinforced cortex of the damaged bleached hair.

TABLE 3*
Plateau Stress Measurements (I)

| | | | | | Composition |
|---|---|---|---|---|---|
| | | | | | C (MA-CDI) + |
| | | | Polycarbodiimide | Composition | flat-iron |
| Plateau | | Carboxylic | 1 | C (MA-CDI) | (FI) |
| stress (MPa) | No treatment | acid 1 (MA) | (CDI) | (Protocol A) | (Protocol B) |
| mean ± | 121.2 ± 13.7 | 130.8 ± 12.2 | 133.7 ± 25.8 | 135.6 ± 18.7 | 173.5 ± 24.9 |
| std. dev. | | | | | |
| p-value**- | — | .112 | .016 | .003 | .000 |
| Untreated vs. | | | | | |
| Others | | | | | |
| p-value-MA | .112 | — | .953 | .747 | .000 |
| vs. Others | | | | | |
| p-value-CDI | .016 | .953 | — | .988 | .000 |
| vs. Others | | | | | |
| p-value- | .003 | .747 | .988 | — | .000 |
| MACDI vs. | | | | | |
| Others | | | | | |
| p-value- | .000 | .000 | .000 | .000 | — |
| MACDIFI vs. | | | | | |
| Others | | | | | |

*Multiple Comparisons Table-Tukey HSD (Use SPSS Software)
**a p-value of less than 0.05 indicates that the difference is statistically significant.

Summary of Findings:

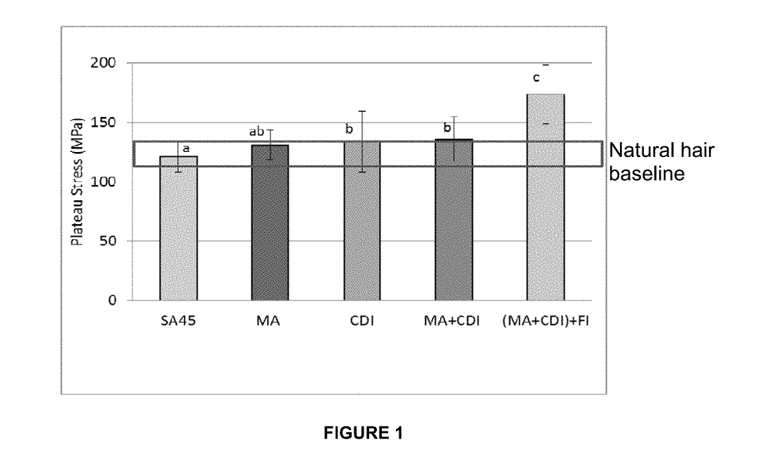

It was observed that the treatment of the hair with the composition C resulted in higher plateau stress values as compared to the values obtained from the treatment of the hair with the untreated composition. See also, FIG. 1.

It was also observed that the treatment of the hair with the composition C in conjunction with the use of heat (flat iron) resulted in higher plateau stress values as compared to the values obtained from the treatment of the hair with the untreated and compositions A and B.

While the treatment with composition B (polycarbodiimide alone) resulted in a statistical difference when compared to no treatment on the hair, the mean plateau stress value was still significantly lower than the mean value obtained from the treatment with Composition C in conjunction with heat.

A significant increase of plateau stress value indicates that the hair fibers have a greater energy absorption capability. Therefore, the results indicate that the treatment of the hair with the composition C which contains both polycarbodiimide and carboxylic acid, especially in conjunction with heating the hair (as in the flat-ironing step), imparted greater mechanical strength to bleach damaged hair.

TABLE 4*
Break Strength Measurements (II)

| | | | | | Composition |
|---|---|---|---|---|---|
| | | | | | C (MA- |
| | | | | Composition | CDI) + flat- |
| | | | | C (MA- | iron (FI) |
| Break stress | No | Carboxylic | Polycarbodiimide | CDI) | (Protocol |
| (MPa) | treatment | acid 1 (MA) | 1 (CDI) | (Protocol A) | B) |
| mean ± | 174.0 ± 36.1 | 195.6 ± 36.1 | 190.1 ± 44.2 | 197.9 ± 38.9 | 252.8 ± 51.1 |
| std. dev. | | | | | |
| p-value- | — | .075 | .303 | .036 | .000 |
| Untreated** | | | | | |
| vs. Others | | | | | |
| p-value-MA | .075 | — | .965 | .999 | .000 |
| vs. Others | | | | | |
| p-value-CDI | .303 | .965 | — | .883 | .000 |
| vs. Others | | | | | |
| p-value- | .036 | .999 | .883 | — | .000 |
| MACDI vs. | | | | | |
| Others | | | | | |
| p-value- | .000 | .000 | .000 | .000 | — |
| MACDIFI vs. | | | | | |
| Others | | | | | |

*Multiple Comparisons Table-Tukey HSD (Use SPSS Software)
**a p-value of less than 0.05 indicates that the difference is statistically significant.

Summary of Findings:

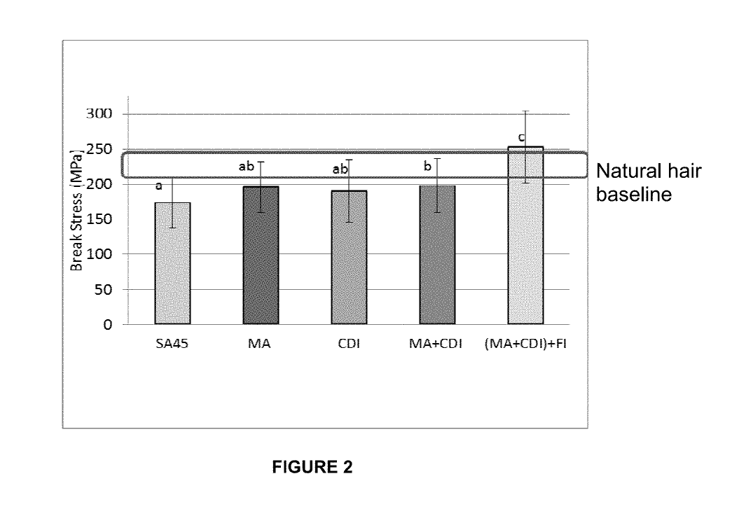

It was observed that the treatment of the hair with the composition C resulted in higher break stress values as compared to the values obtained from the treatment of the hair with the untreated composition. See also, FIG. 2.

It was also observed that the treatment of the hair with the composition C in conjunction with the use of heat (flat iron) resulted in higher plateau stress values as compared to the values obtained from the treatment of the hair with the untreated and compositions A and B.

A significant increase of break stress value indicates that the hair fibers have a reinforced or stronger cortex. Therefore, the results indicate that the treatment of the hair with the composition C which contains both polycarbodiimide and carboxylic acid, especially in conjunction with heating the hair (as in the flat-ironing step), imparted greater mechanical strength to bleach damaged.

TABLE 5*
Toughness Measurements

| | | | | | Composition |
|---|---|---|---|---|---|
| | | | | | C (MA- |
| | | | | | CDI) + flat- |
| | | | Polycarbodiimide | Composition | iron (FI) |
| Toughness | No | Carboxylic | 1 | C (MA-CDI) | (Protocol |
| (10⁷ J/m3) | treatment | acid 1 (MA) | (CDI) | (Protocol A) | B) |
| mean ± | 5.54 ± 2.1 | 6.42 ± 2.2 | 6.12 ± 2.3 | 6.26 ± 1.8 | 7.84 ± 2.8 |
| std. dev. | | | | | |
| p-value- | — | .289 | .703 | .508 | .000 |
| Untreated** | | | | | |
| vs. Others | | | | | |
| p-value-MA | .289 | — | .961 | .996 | .016 |
| vs. Others | | | | | |
| p-value-CDI | .703 | .961 | — | .998 | .002 |
| vs. Others | | | | | |
| p-value- | .508 | .996 | .998 | — | .005 |
| MACDI vs. | | | | | |
| Others | | | | | |
| p-value- | .000 | .016 | .002 | .005 | — |
| MACDIFI vs. | | | | | |
| Others | | | | | |

*Multiple Comparisons Table-Tukey HSD (Use SPSS Software)
**a p-value of less than 0.05 indicates that the difference is statistically significant.

Summary of Findings:

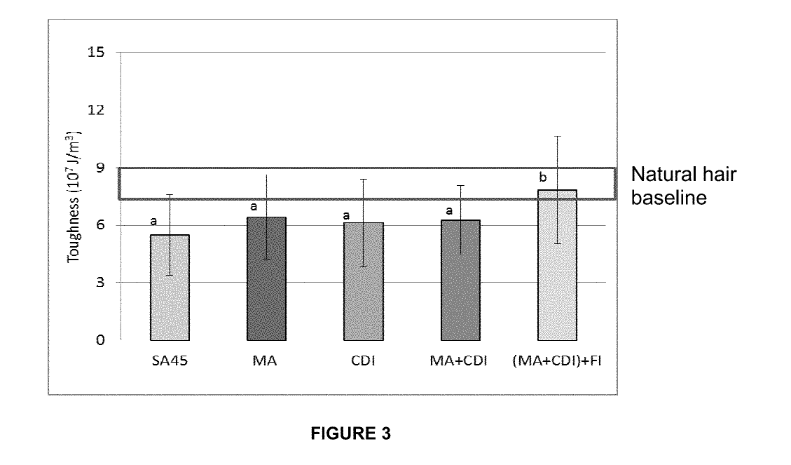

It was observed that the treatment of the hair with the composition C, in conjunction with heating the hair (as in the flat-ironing step), resulted in higher toughness values as compared to the values obtained from the treatment of the hair with the other compositions. See also, FIG. 3.

A significant increase of toughness value indicates that the hair fibers have a reinforced or stronger cortex. Therefore, the results indicate that the treatment of the hair with the composition C which contains both polycarbodiimide and carboxylic acid, especially in conjunction with heating the hair (as in the flat-ironing step), imparted greater mechanical strength to bleach damaged.

While the invention has been described with reference to certain exemplary or preferred embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A composition for treatment of keratinous fibers comprising:
at least one polycarbodiimide compound; and
at least one carboxylic acid selected from the group consisting of maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof.

2. The composition according to claim 1, wherein the at least one polycarbodiimide compound has the following formula: 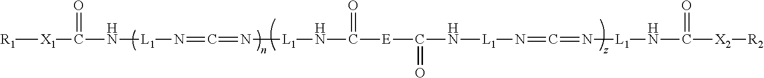
wherein X₁ and X₂, each independently, represents O, S or NH; R₁ and R₂, each independently, a hydrocarbon group containing one or more catenary or non-catenary hetero-atoms and containing linear or branched and cyclic or acyclic groups which are ionic or non-ionic segments or a partially or fully fluorinated hydrocarbon group containing one or more catenary or non-catenary hetero-atoms; n and z are, each independently, an integer of 0 to 20; L₁ represents a C₁ to C₁₈ divalent aliphatic hydrocarbon group, a C₃ to C₁₃ divalent alicyclic hydrocarbon group, a C₆ to C₁₄ divalent aromatic hydrocarbon group, a C₃ to C₁₂ divalent heterocyclic group, or a C₆ to C₁₄ divalent aromatic hydrocarbon group, wherein a plurality of L₁ groups may be identical to or different from one another; E is a radical selected from:
O—R₃—; S—R₄—S; and
R₅—N—R₄—N—R₅;
wherein R₃ and R₄ are, each independently, hydrocarbon radicals that may contain halogen atoms or one or more catenary or non-catenary hetero atoms, including an aromatic, cycloaliphatic, aryl and linear or branched alkyl radical and R₅ is hydrogen or a hydrocarbon radical, the hydrocarbon radical, when present, includes halogen atoms or one or more catenary or non-catenary hetero atoms.

3. The composition according to claim 1, wherein the at least one polycarbodiimide compound is a co-polymer derived from alpha-methylstyryl-isocyanates having the following formula: 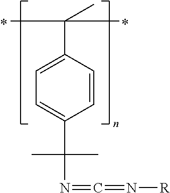
wherein R is an alkyl, cycloalkyl or aryl group having from 1 to 24 carbon atoms.

4. The composition according to claim 1, wherein the at least one polycarbodiimide compound is a compound having the following structure: 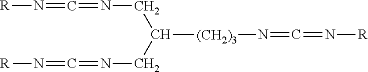
wherein R is an alkyl, cycloalkyl or aryl group.

5. The composition according to claim 4, wherein R is an alkyl, cycloaklyl or aryl group having from 1 to 24 carbon atoms.

6. The composition according to claim 1, the composition includes amounts of each of the polycarbodiimide compound and the carboxylic acid sufficient to impart to the keratinous fibers after application thereto, one or more of:
improved conditioning;
protection from damage caused by extrinsic and/or intrinsic factors;
repair when the keratinous fibers are in a damaged condition;
minimized or no breakage;
increased or improved mechanical strength;
increased hydrophobicity; or
increased hold to the shape or configuration of the fibers.

7. The composition according to claim 2, wherein the at least one polycarbodiimide compound is present in a concentration, by weight, of from about 0.1% to about 10%, based on the total weight of the composition.

8. The composition according to claim 2, wherein the polycarbodiimide compound is present in a concentration, by weight, of from about 0.2% to about 5%, based on the total weight of the composition.

9. The composition according to claim 2, wherein the at least one carboxylic acid is present in a concentration, by weight, of from about 0.25% to about 20%, based on the total weight of the composition.

10. The composition according to claim 2, wherein the at least one carboxylic acid is present in a concentration, by weight, of from about 0.4% to about 10%, based on the total weight of the composition.

11. The composition according to claim 2, wherein the at least one carboxylic acid is present in an equal proportion to or greater proportion than the polycarbodiimide compound.

12. The composition according to claim 2, wherein the carboxylic acid is maleic acid.

13. The composition according to claim 1, comprising a solvent selected from water, organic solvents, and a mixture thereof.

14. The composition according to claim 1, comprising at least one additive chosen from surfactants (anionic, nonionic, cationic and amphoteric/zwitterionic), rheology modifiers, thickening and/or viscosity modifying agents, nacreous agents, opacifiers, vitamins, preserving agents, pH stabilizers, and mixtures thereof.

15. The composition according to claim 2, wherein the keratinous fibers include hair, and the composition is heat-activated after or during its application onto the hair.

16. The composition according to claim 2, wherein the keratinous fibers include hair, and wherein the composition is formed in-situ on the hair from a step-wise application on the hair of the at least one polycarbodiimide compound and the at least one carboxylic acid which are each separately contained in a solvent chosen from water, organic solvent, or mixtures thereof.

17. A method of treating keratinous fibers, comprising applying onto keratinous fibers, a composition containing:
at least one polycarbodiimide compound; and
at least one carboxylic acid selected from the group consisting of maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof.

18. The method according to claim 17, comprising a step of heating the fibers during or before or after the composition is applied onto the fibers.

19. The method according to claim 18, comprising crimping or smoothing the fibers during the heating step using a flat iron device at a temperature of between about 135° C. and about 250° C.

20. The method according to claim 18, comprising brushing or combing the fibers during the heating step wherein the heat is applied using a blow dryer device set at a temperature of between about 30° C. and about 100° C.

21. The method according to claim 17, wherein the at least one polycarbodiimide compound is present in a concentration, by weight, of from about 0.25% to about 20% and the at least one carboxylic acid is present in a concentration, by weight, of from about 0.25% to about 20%, all weights being based on the total weight of the composition; and
wherein the at least one polycarbodiimide compound has the following formula: 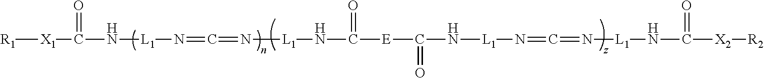
wherein X₁ and X₂, each independently, represents O, S or NH; R₁ and R₂, each independently, a hydrocarbon group containing one or more catenary or non-catenary hetero-atoms and containing linear or branched and cyclic or acyclic groups which are ionic or non-ionic segments or a partially or fully fluorinated hydrocarbon group containing one or more catenary or non-catenary hetero-atoms; n and z are, each independently, an integer of 0 to 20; L₁ represents a C₁ to C₁₈ divalent aliphatic hydrocarbon group, a C₃ to C₁₃ divalent alicyclic hydrocarbon group, a C₆ to C₁₄ divalent aromatic hydrocarbon group, a C₃ to C₁₂ divalent heterocyclic group, or a C₆ to C₁₄ divalent aromatic hydrocarbon group, wherein a plurality of L₁ groups may be identical to or different from one another; E is a radical selected from:
O—R₃—O; S—R₄—S; and
R₅—N—R₄—N—R₅;
wherein R₃ and R₄ are, each independently, hydrocarbon radicals that may contain halogen atoms or one or more catenary or non-catenary hetero atoms, including an aromatic, cycloaliphatic, aryl and linear or branched alkyl radical and R₅ is hydrogen or a hydrocarbon radical, the hydrocarbon radical, when present, includes halogen atoms or one or more catenary or non-catenary hetero atoms.

22. The method according to claim 17, comprising a step-wise application on the fibers of the at least one polycarbodiimide compound and the at least one carboxylic acid which are each separately contained in a solvent chosen from water, organic solvent, or mixtures thereof; and wherein the composition is formed in-situ on the fibers.
